(19) United States
(12) Patent Application Publication
(10) Pub. No.: US 2024/0392370 A1
(43) Pub. Date: Nov. 28, 2024

(54) METHOD FOR DESIGNING MULTIPLEX PCR PRIMERS BASED ON ITERATION AND COMPUTER DEVICE

(71) Applicants: CHENGDU BOE OPTOELECTRONICS TECHNOLOGY CO., LTD., Sichuan (CN); BOE TECHNOLOGY GROUP CO., LTD., Beijing (CN)

(72) Inventor: Mengjia LIU, Beijing (CN)

(73) Assignees: CHENGDU BOE OPTOELECTRONICS TECHNOLOGY CO., LTD., Sichuan (CN); BOE TECHNOLOGY GROUP CO., LTD., Beijing (CN)

(21) Appl. No.: 18/014,898

(22) PCT Filed: Oct. 29, 2021
(86) PCT No.: PCT/CN2021/127600

§ 371 Date: Jan. 6, 2023

(51) Int. Cl. C12Q 1/6876; G16B 25/20

(52) U.S. Cl. CPC C12Q 1/6876; G16B 25/20; C12Q 2600/16

(57) ABSTRACT

The present disclosure provides a method for designing multiplex polymerase chain reaction (PCR) primers based on iteration and a computer device. The method for designing multiplex PCR primers based on iteration includes: acquiring at least one target interval of one of DNA to be tested and RNA to be tested, a plurality of sub-intervals in a target interval and at least one avoidance region in the target interval; performing iterative design of PCR primers, avoiding the at least one avoidance region, for the plurality of sub-intervals to generate primers; filtering and screening the generated primers according to a preset filter condition to obtain target primers; and combining the obtained target primers to generate a primer pool.

### CROSS-REFERENCE TO RELATED APPLICATION

This application is a national phase entry under 35 USC 371 of International Patent Application No. PCT/CN2021/127600, filed on Oct. 29, 2021, which is incorporated herein by reference in its entirety.

### TECHNICAL FIELD

The present disclosure relates to the field of polymerase chain reaction (PCR) primer design, and in particular, to a method for designing multiplex PCR primers based on iteration and a computer device.

### BACKGROUND

Polymerase chain reaction (PCR) technology, also known as in vitro gene amplification technology, is a widely used molecular biology technology at present. PCR technology is a technology for synthesizing a large amount of specific genes in vitro or in a test-tube by using DNA polymerase, and the basic working principle of PCR technology is that a DNA molecule to be amplified is used as a template, a pair of oligonucleotide fragments that are complementary with the template are used as primers, and due to the action of DNA polymerase, the primers extend along the template chain according to the base complementary pairing principle until a new DNA synthesis is completed. By repeating the process continuously, a target DNA fragment may be amplified. With the development of PCR technology, a variety of related technologies have emerged. Multiplex PCR technology has been used to rapidly detect deletion of exons of genes associated with human Duchenne muscular dystrophy as early as 1988, which may greatly improve efficiency of PCR. In addition, such PCR technology that provides two or more pairs of primers in the same PCR reaction system can amplify a plurality of nucleotide fragments simultaneously.

The steps of a current conventional method for designing multiplex primers are as follows: 1) designing all primers at one time in the target region; 2) performing filtration after designing; 3) screening a target primer pool after filtration. The method has problems such as unreasonable selection of target intervals, large number of low-quality primers, filtration of a large number of primers, and redesign due to complete filtration of primers in some target intervals.

### SUMMARY

In an aspect, a method for designing multiplex polymerase chain reaction (PCR) primers based on iteration is provided. The method for designing multiplex PCR primers based on iteration includes: acquiring at least one target interval of one of DNA to be tested and RNA to be tested, a plurality of sub-intervals in a target interval and at least one avoidance region in the target interval; performing iterative design of PCR primers, avoiding the at least one avoidance region, for the plurality of sub-intervals to generate primers; filtering and screening the generated primers according to a preset filter condition to obtain target primers; and combining the obtained target primers to generate a primer pool.

In some embodiments, acquiring the at least one avoidance region in the target interval, includes: acquiring flanking information of each sub-interval in the target interval, the flanking information including at least one of guanine-cytosine (GC) contents, single nucleotide variants (SNVs) and complexities of upstream and downstream sequences of the sub-interval; and determining a part of the plurality of sub-intervals as the at least one avoidance region in the target interval according to the flanking information.

In some embodiments, determining a part of the plurality of sub-intervals as the at least one avoidance region in the target interval according to the flanking information, includes: if at least one of the upstream sequence and the downstream sequence has a GC content greater than 75% or less than 35%, determining a sub-interval where the at least one of the upstream sequence and the downstream sequence is located as an avoidance region in the target interval; if at least one of the upstream sequence and the downstream sequence has an acquired SNV, a sub-interval where the at least one of the upstream sequence and the downstream sequence is located being a mutation region, and determining the mutation region as an avoidance region in the target interval; and if at least one of the upstream sequence and the downstream sequence has a complexity lower than a preset complexity threshold, determining a sub-interval where the at least one of the upstream sequence and the downstream sequence is located as an avoidance region in the target interval.

In some embodiments, acquiring the plurality of sub-intervals in the target interval, includes: acquiring a length of each target interval according to a preset amplification length of the primers; and comparing the length of the target interval with the preset amplification length; if the length of the target interval is greater than the preset amplification length, acquiring a number of sub-intervals to be split from the target interval, and acquiring a range of a number of primers of a target fragment in the target interval.

In some embodiments, after comparing the length of the target interval with the preset amplification length, the method further includes: if the length of the target interval is less than or equal to the preset amplification length, determining that the target interval is amplified by using a pair of primers.

In some embodiments, performing the iterative design of PCR primers, avoiding the at least one avoidance region, for the plurality of sub-intervals to generate the primers, includes: according to a position of an avoidance region and flanking information of the target interval, determining whether a sub-interval is adjacent to the avoidance region; if the sub-interval is adjacent to the avoidance region, shifting the sub-interval to skip the avoidance region; and if the sub-interval is not adjacent to the avoidance region, maintaining a position of the sub-interval.

In some embodiments, filtering and screening the generated primers according to the preset filter condition to obtain the target primers, includes: aligning the generated primers to a genome to sort binding sites of the primers and the genome in reverse order and screen out a primer pair with least non-specific binding in each sub-interval, so as to obtain remaining primers after screening; filtering dimers in the remaining primers in the same amplification interval directly, and labeling dimers in the remaining primers in different amplification intervals.

In some embodiments, combining the obtained target primers to generate the primer pool, includes: selecting primers within a preset temperature range according to the obtained target primers; splitting a combination of primers with dimer mutual exclusion into different primer pools; and selecting primers without mutual exclusion for combination to generate the final primer pool.

In some embodiments, the preset temperature range includes an optimal temperature range and a sub-optimal temperature range.

In another aspect, a computer device is provided. The computer device includes a memory and a processor. The memory has stored therein a computer program that capable of being run on the processor. When executing the computer program, the processor performs: acquiring at least one target interval of one of DNA to be tested and RNA to be tested, a plurality of sub-intervals in a target interval, and at least one avoidance region in the target interval; receiving the at least one avoidance region in the target interval, and performing iterative design of PCR primers, avoiding the at least one avoidance region, for the plurality of sub-intervals to generate primers; receiving the generated primers, and filtering and screening the primers according to a preset filtering condition to obtain target primers; and receiving the obtained target primers, and combining the target primers to generate a primer pool.

In some embodiments, when executing the computer program, the processor further performs: acquiring flanking information of each sub-interval in the target interval, the flanking information including at least one of guanine-cytosine (GC) contents, single nucleotide variants (SNVs) and complexities of upstream and downstream sequences of the sub-interval; and receiving the flanking information, and determining a part of the plurality of sub-intervals as the at least one avoidance region in the target interval according to the flanking information.

In some embodiments, when executing the computer program, the processor further performs: if at least one of the upstream sequence and the downstream sequence has a GC content greater than 75% or less than 35%, determining a sub-interval where the at least one of the upstream sequence and the downstream sequence is located as an avoidance region in the target interval; if at least one of the upstream sequence and the downstream sequence has an acquired SNV, determining a sub-interval where the at least one of the upstream sequence and the downstream sequence is located being a mutation region, and determining the mutation region as an avoidance region in the target interval; and if at least one of the upstream sequence and the downstream sequence has a complexity lower than a preset complexity threshold, determining a sub-interval where the at least one of the upstream sequence and the downstream sequence is located as an avoidance region in the target interval.

In some embodiments, when executing the computer program, the processor further performs: acquiring a length of each target interval according to a preset amplification length of the primers; comparing the length of the target interval with the preset amplification length; and if the length of the target interval is greater than the preset amplification length, acquiring a number of sub-intervals to be split from the target interval, and acquiring a range of a number of primers of a target fragment in the target interval.

In some embodiments, when executing the computer program, the processor further performs: after comparing the length of the target interval with the preset amplification length, if the length of the target interval is less than or equal to the preset amplification length, determining that the target interval is amplified by using a pair of primers.

In some embodiments, when executing the computer program, the processor further performs: performing the iterative design of PCR primers for the plurality of sub-intervals; determining whether a sub-interval is adjacent to an avoidance region according to a position of the avoidance region and flanking information of the target interval; shifting the sub-interval to skip the avoidance region if the sub-interval is adjacent to the avoidance region; and maintaining a position of the sub-interval if the sub-interval is not adjacent to the avoidance region.

In some embodiments, when executing the computer program, the processor further performs: aligning the generated primers to a genome to sort binding sites of the primers and the genome in reverse order and screen out a primer pair with least non-specific binding in each sub-interval, so as to obtain remaining primers after screening; and filtering dimers in the remaining primers in the same amplification interval directly, and labeling dimers in the remaining primers in different amplification intervals.

In some embodiments, when executing the computer program, the processor further performs: screening a temperature range adapted to the obtained target primers; and distinguishing a combination of primers with dimer mutual exclusion and splitting the combination of primers with dimer mutual exclusion into different primer pools.

In yet another aspect, a non-transitory computer-readable storage medium is provided. The non-transitory computer-readable storage medium has stored therein a computer program that, when executed by a processor, causes the processor to perform the method as described in any of the above embodiments.

### BRIEF DESCRIPTION OF THE DRAWINGS

In order to describe technical solutions in the present disclosure more clearly, accompanying drawings to be used in some embodiments of the present disclosure will be introduced briefly below. Obviously, the accompanying drawings to be described below are merely accompanying drawings of some embodiments of the present disclosure, and a person of ordinary skill in the art may obtain other drawings according to these drawings. In addition, the accompanying drawings in the following description may be regarded as schematic diagrams, but are not limitations to actual sizes of products, actual processes of methods or actual timings of signals to which the embodiments of the present disclosure relate.

FIG. 1 is a primer avoidance design diagram of target intervals of multiplex primers design, provided by embodiments of the present disclosure;

FIG. 2 is a primer flow diagram of an iterative design for multiplex primers, provided by embodiments of the present disclosure;

FIG. 3 is a design diagram of an amplification temperature screening scheme for a multiplex primer pool, provided by embodiments of the present disclosure;

FIG. 4 is a block diagram of a system for designing multiplex polymerase chain reaction (PCR) primers, provided by embodiments of the present disclosure;

FIG. 5 is a block diagram of an acquisition module, provided by embodiments of the present disclosure;

FIG. 6 is a block diagram of an avoidance sub-module, provided by embodiments of the present disclosure;

FIG. 7 is a block diagram of a primer generation module, provided by embodiments of the present disclosure;

FIG. 8 is a block diagram of a filtering and screening module, provided by embodiments of the present disclosure;

FIG. 9 is a block diagram of another system for designing multiplex PCR primers, provided by embodiments of the present disclosure; and

FIG. 10 is a block diagram of a computer device, provided by embodiments of the present disclosure.

### DETAILED DESCRIPTION

Technical solutions in some embodiments of the present disclosure will be described clearly and completely below with reference to the accompanying drawings. Obviously, the described embodiments are merely some but not all embodiments of the present disclosure. All other embodiments obtained by a person of ordinary skill in the art based on embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive sense, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, specific features, structures, materials or characteristics may be included in any one or more embodiments or examples in any suitable manner.

In an aspect, the embodiments of the present disclosure provide a method for designing multiplex polymerase chain reaction (PCR) primers based on iteration. The method for designing multiplex PCR primers based on iteration includes: acquiring target interval(s) of DNA to be tested or RNA to be tested, a plurality of sub-intervals in a target interval and avoidance region(s) in the target interval, the target interval(s) of the DNA including sequence information and position information of the DNA, and the target interval(s) of the RNA including sequence information and position information of the RNA; performing iterative design of PCR primers, avoiding the avoidance region, for the plurality of sub-intervals to generate primers; filtering and screening the generated primers according to a preset filter condition to obtain target primers; and combining the obtained target primers to generate a primer pool.

In the method for designing multiplex PCR primers based on iteration provided by the embodiments of the present disclosure, the avoidance region(s) in the target interval are acquired by adding a preprocessing step of the target interval, so as to avoid the avoidance region(s), which may avoid a problem of unreasonable selection of the target interval. The iterative design of PCR primers is adopted, so that a primer interval designed each time is based on a successful design of a previous interval, which may ensure effectiveness of design and reduce generation of primers with low quality and strong background noise. The generated primers are, according to the preset filter condition, filtered, screened and combined, so as to generate the primer pool.

In some embodiments of the present disclosure, acquiring the avoidance region(s) in the target interval, includes: acquiring flanking information of each sub-interval in the target interval, the flanking information including at least one of guanine-cytosine (GC) contents, single nucleotide variants (SNVs) and complexities of upstream and downstream sequences of the sub-interval; and determining a part of the plurality of sub-intervals as the avoidance region(s) in the target interval according to the flanking information.

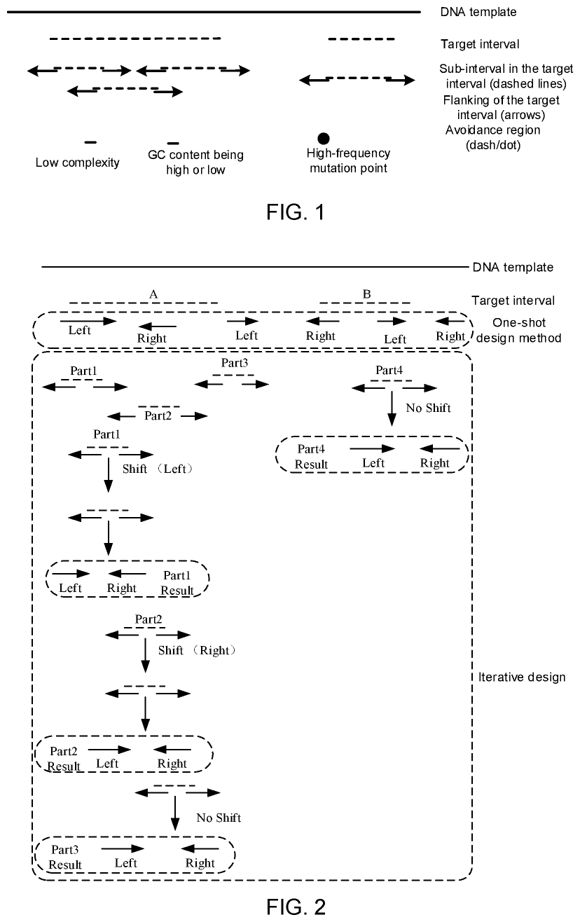

Based on the above contents, with reference to FIG. 1, determining the part of the plurality of sub-intervals as the avoidance region(s) in the target interval according to the flanking information, includes: if the upstream sequence and/or the downstream sequence has the GC content greater than 75% or less than 35%, determining a sub-interval where the upstream sequence is located and/or a sub-interval where the downstream sequence is located as the avoidance region in the target interval, so that the avoidance region is avoided during primer design; if the SNV of the upstream sequence and/or the SNV of the downstream sequence is obtained, a sub-interval where the upstream sequence is located and/or a sub-interval where the downstream sequence is located being a mutation region, and determining the mutation region as the avoidance region in the target interval, so that the avoidance region is avoided during primer design; and if the upstream sequence and/or the downstream sequence has the complexity lower than a preset complexity threshold, determining a sub-interval where the upstream sequence is located and/or a sub-interval where the downstream sequence is located as the avoidance region in the target interval, so that the avoidance region is avoided during primer design.

In some embodiments, acquiring the plurality of sub-intervals in the target interval, includes: acquiring a length of each target interval according to a preset amplification length of the primers; and comparing the length of the target interval with the preset amplification length; if the length of the target interval is greater than the preset amplification length, acquiring the number of sub-intervals to be split from the target interval, and acquiring a range of the number of primers of a target fragment in the target interval.

In some embodiments, after comparing the length of the target interval with the preset amplification length, the method further includes: if the length of the target interval is less than or equal to the preset amplification length, determining that the target interval is amplified by using a pair of primers.

In some embodiments of the present disclosure, performing the iterative design of PCR primers, avoiding the avoidance region, for the plurality of sub-intervals to generate the primers, includes: according to a position of the avoidance region and the flanking information of the target interval, shifting the sub-interval to skip the avoidance region if the sub-interval is adjacent to the avoidance region, and maintaining a position of the sub-interval if the sub-interval is not adjacent to the avoidance region.

Based on the above contents, referring to FIG. 2, the iterative design of PCR primers is performed on each sub-interval. According to the position of the avoidance region and the flanking information of the target interval, the sub-interval is shifted. The situation depends on whether the avoidance region is adjacent to the sub-interval or not. The sub-interval is not shifted in a case where the avoidance region is not adjacent to the sub-interval, and the sub-interval is shifted in a case where the avoidance region is adjacent to the sub-interval, so as to avoid the avoidance region. There may be left shift (shift left), right shift (shift right), and no shift during shift, and pairs of primers (Part 1/Part 2/Part 3/Part 4 Result) are designed after shifting. Primer pairs located at a 3′ end of a sub-interval are identified with “Left” and primer pairs located at a 5′ end of a sub-interval are identified with “Right”. For example, in a sub-interval Part 1, the avoidance region which needs to be avoided or a re-planed region is determined and designed according to the existing sub-intervals and the flanking information, left shift (shift left) is concluded comprehensively, and primers are generated after shifting. In this way, effectiveness and high specificity of this design may be ensured. After this design succeeds, an iterative design is continued, so as to ensure the effectiveness and the high specificity of each design.

In FIG. 2, three pairs of primers are designed through a conventional one-shot design method by comparing and designing a target interval A with a target interval B. The number of the obtained sub-intervals in consideration of target interval information and the flanking information in the iterative design of the method of the embodiments of the present disclosure may be equal to or beyond the conventional number of target intervals. Due to the target interval and the flanking information, information of a design position after shifting is inconsistent with information of a position in a conventional design. Even if there is no shift, the information of the design position may also be inconsistent with the information of the position in the conventional design due to preprocessed avoidance information. Such a design allows the design position to be at a position that is accurate and has high specificity. In the design method based on iteration, a next new design is based on an existing design, so that the design position may be in a reasonable position.

Moreover, in addition to the above design thinking, primer design software completes design operations by calling primer 3 and other auxiliary design software. This step includes providing required information, which includes a length range of an amplified fragment, a temperature of amplification experiment (Tm, GC and ion concentration), etc., so that a primer sequence of each sub-interval may be obtained.

In some embodiments of the present disclosure, filtering and screening the generated primers according to the preset filter condition to obtain the target primers, includes: aligning the generated primers to a genome to sort binding sites of the primers and the genome in reverse order and screen out a primer pair with the least non-specific binding in each sub-interval, so as to acquire remaining primers after screening; and filtering dimers in the remaining primers in the same amplification interval directly, and labeling dimers in the remaining primers in different amplification intervals.

Based on the above contents, the generated primers are aligned to the genome, and information of all successful alignments of each primer on the genome is obtained. These alignments include specific alignments (successful alignments with a target interval) and non-specific alignments (not successful alignments with the target interval but successful alignments with other regions). These alignment results each include the following information: a position of alignment on the genome (chromosome, start, end, an alignment length, the number of mismatches, the number of Gaps, etc.). According to the alignment results, the alignment results are counted, and the numbers of specific alignments and non-specific alignments of each primer are counted. If there are few non-specific alignments, that is, there are few non-specific amplification binding sites, it indicates that background noise is low and a primer effect is good. The reverse order of the binding sites (e.g., the numbers of binding sites of the three primers is sorted as 1000, 30 and 1 in reverse order) is obtained by sorting the numbers of the binding sites, so as to filter the primers according to the above criteria.

In some embodiments of the present disclosure, combining the obtained target primers to generate the primer pool, includes: selecting primers within a preset temperature range according to the obtained target primers; splitting a combination of primers with dimer mutual exclusion into different primer pools, and selecting primers without mutual exclusion for combination to generate the final primer pool.

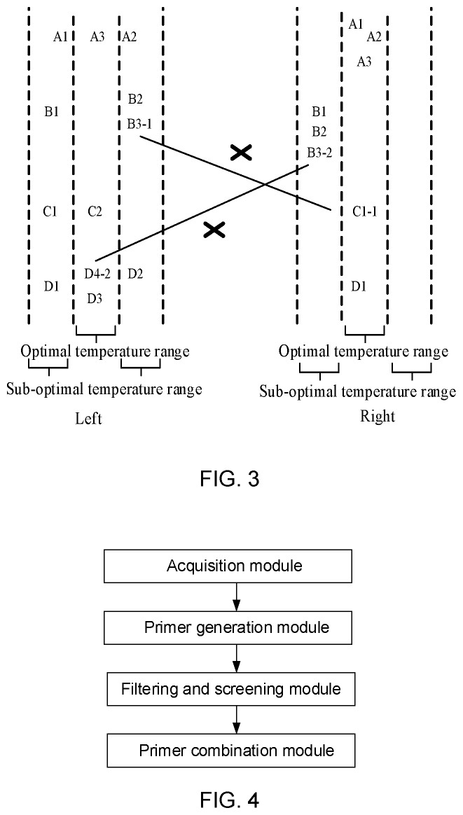

Referring to FIG. 3, a temperature range required for a specific experiment is divided into two parts including an optimal temperature range and a sub-optimal temperature range. Primers for each sub-interval (ABCD) may exist that all are within the optimal temperature range (Right A), a part are within the optimal temperature range and the other part are within the sub-optimal temperature range (Left A), or all are within the sub-optimal temperature range (Left B). In the absence of other influence conditions (e.g., dimer mutual exclusion), primers within the optimal temperature range, such as Left A3 or Right A1/A2/A3, are preferably selected.

For combinations of primers with dimer mutual exclusion, primer pairs labeled as mutually exclusive pairs (e.g., Left B3-1 and Right C1-1, Left D4-2 and Right B3-2) are split into different primer pools, and then in a case where temperature ranges of pairs of primers are similar (Right B), it is preferred to select primers without mutual exclusion (Right B1 or Right B2). In summary, primers within the optimal temperature range are preferably selected and, if there are not primers within the optimal temperature range, primers within the sub-optimal temperature range are considered, and then primers with mutual exclusion are split into different primer pools (the primers with mutual exclusion, such as examples marked with X in FIG. 3, are split into different primer pools).

In another aspect, a system for designing multiplex PCR primers based on iteration is provided. As shown in FIG. 4, the system for designing multiplex PCR primers based on iteration includes an acquiring module, a primer generation module, a filtering and screening module and a primer combination module. The acquisition module is used to acquire target interval(s) of DNA to be tested or RNA to be tested, a plurality of sub-intervals in a target interval and avoidance region(s) in the target interval. The primer generation module is used to receive the avoidance region(s) in the target interval provided by the acquisition module, and perform iterative design of PCR primers, avoiding the avoidance region(s), for the plurality of the sub-intervals to generate primers. The filtering and screening module is used to receive the primers generated by the primer generation module, and filter and screen the primers according to a preset filtering condition to obtain target primers. The primer combination module is used to receive the target primers provided by the filtering and screening module, and combine the obtained target primers to generate a primer pool.

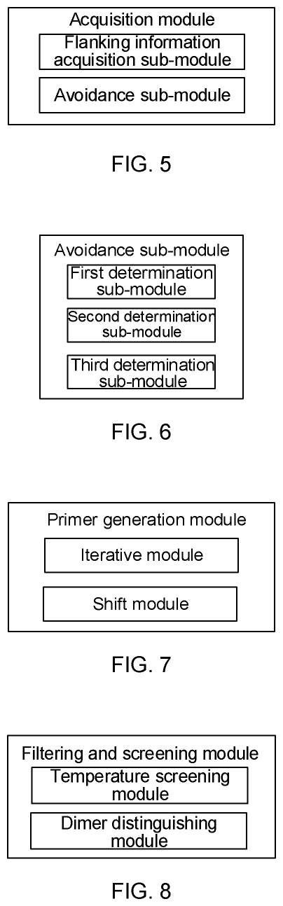

In some embodiments of the present disclosure, as shown in FIG. 5, the acquisition module includes a flanking information acquisition sub-module and an avoidance sub-module. The flanking information acquisition sub-module is used to acquire flanking information of each sub-interval in the target interval. The flanking information includes at least one of GC contents, SNVs and complexities of upstream and downstream sequences of the sub-interval. The avoidance sub-module is used to receive the flanking information provided by the flanking information acquisition sub-module, and determine a part of the plurality of sub-intervals as the avoidance region(s) in the target interval according to the flanking information.

In some embodiments, as shown in FIG. 6, the avoidance sub-module includes a first determination sub-module, a second determination sub-module and a third determination sub-module. The first determination sub-module is used to, if the upstream sequence and/or the downstream sequence has the GC content greater than 75% or less than 35%, determine a sub-interval where the upstream sequence is located and/or a sub-interval where the downstream sequence is located as the avoidance region in the target interval. The second determination sub-module is used to, if the SNV of the upstream sequence and/or the SNV of the downstream sequence is obtained, a sub-interval where the upstream is located and/or a sub-interval where the downstream sequence is located being a mutation region, and determine the mutation region as the avoidance region in the target interval. The third determination sub-module is used to, if the upstream sequence and/or the downstream sequence has the complexity lower than a preset complexity threshold, determine a sub-interval where the upstream sequence is located and/or a sub-interval where the downstream sequence is located as the avoidance region in the target interval.

In some embodiments, as shown in FIG. 7, the primer generation module includes an iterative module and a shift module. The iterative module is used to perform the iterative design of PCR primers for the plurality of sub-intervals. The shift module is used to, according to a position of the avoidance region and the flanking information of the target interval, shift a sub-interval to skip the avoidance region if the sub-interval is adjacent to the avoidance region; and maintain a position of the sub-interval if the sub-interval is not adjacent to the avoidance region.

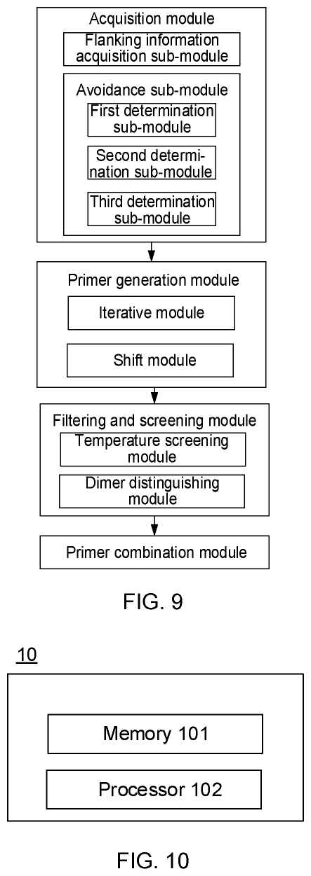

In some embodiments, as shown in FIG. 8, the filtering and screening module includes a temperature screening module and a dimer distinguishing module. The temperature screening module is used to screen a temperature range adapted to the obtained target primers. The dimer distinguishing module is used to distinguish a combination of primers with dimer mutual exclusion and split the combination of primers with dimer mutual exclusion into different primer pools. FIG. 9 shows the system for designing multiplex PCR primers based on iteration in the embodiments of the present disclosure.

In yet another aspect, a computer device is provided. As shown in FIG. 10, the computer device 10 includes a memory 101 and a processor 102. The memory has stored therein a computer program capable of being run on the processor. The processor implements the method as described in any of the above embodiments when executing the computer program.

Some embodiments of the present disclosure provide a computer-readable storage medium (e.g., a non-transitory computer-readable storage medium). The computer-readable storage medium has stored therein computer program instructions that, when run on a computer (e.g., the processor), cause the computer to perform the method as described in any of the above embodiments.

For example, the computer-readable storage medium may include, but is not limited to, a magnetic storage device (e.g., a hard disk, a floppy disk or a magnetic tape), an optical disk (e.g., a compact disk (CD) or a digital versatile disk (DVD)), a smart card, and a flash memory device (e.g., an erasable programmable read-only memory (EPROM), a card, a stick or a key driver). Various computer-readable storage media described in the present disclosure may represent one or more devices and/or other machine-readable storage media, which are used for storing information. The term “computer-readable storage medium” may include, but is not limited to, wireless channels and various other media capable of storing, containing and/or carrying instructions and/or data.

Beneficial effects of the computer device and the computer-readable storage medium are the same as the beneficial effects of the method as described in the above embodiments, and details will be not repeated here.

In the method for designing multiplex PCR primers based on iteration in the embodiments of the present disclosure, for example, multiplex primers are designed for exons of four genes, KRAS, NRAS, BRAF and PIK3CA.

Coordinate information of a whole exon region of a target gene is obtained through a GRCh38 genome annotation file in a format of General Feature Format version 3 (gff3), and an exon overlapping region is de-duplicated. The exon overlapping region is from an exon crossing portion between different transcripts of the same gene. According to the requirements of a relevant experimenter, the length of the target amplified fragment is in a range of 250 bp to 300 bp, the temperature range thereof is 57° C. to 62° C., and the optimal temperature is 59° C. The obtained target interval is pre-assessed, and both the length and the GC content of the target interval are obtained though calculation according to the above experimental requirements. The number of primers for each target interval is preliminarily calculated. If the length of the target interval is less than the preset amplification length, a pair of primers may be used for amplification; if the length of the target interval exceeds the preset amplification length, the number of the sub-intervals to be split from the target interval is calculated, and the range of the number of primers of the target fragment is determined.

According to both a length and an amplification length range of each sub-interval, the upstream and downstream sequences of each sub-interval are obtained by setting a parameter of a flanking length of the sub-interval as 30 bp, and the GC, the SNV and coverage of upstream and downstream flanking sequence are calculated. If the GC content is too high (greater than 75%) or too low (less than 35%), the sub-interval is not suitable to design primers. The Single Nucleotide Polymorphism Database (dbSNP) is used to calculate the sites with the minimum allele frequency greater than or equal to 0.05. These sites are considered unsuitable for the design of primers due to high-frequency mutation in species. Regions with low complexity, such as a polyN sequence or a microsatellite sequence, are not conducive to primer binding, and thus are not suitable to design primers, either. To sum up, it may be obtained that sites with too high or too low GC content, high-frequency mutation, or low complexity are designed as the avoidance regions in the primer design.

For each sub-interval, primers are designed through an iterative method based on preprocessing results. All sub-interval regions are sorted by chromosome sorting and coordinate sorting (sort-k1, 1n-k2, 2n sample.bed, and the sample.bed is a chromosome position coordinate file of the target sub-interval in this experiment and stored in a bed format) to obtain the sorted target sub-intervals. The primers are iteratively designed for the sorted regions. If there is a region that needs to be avoided in a primer design region, the primer design region is shifted left firstly to search a region without an avoidance point. If the region is found, the design is started. If the region is not found, the primer design region is shifted right from a starting position (a position before shifting left) to search a region without the avoidance point. If the region is found, the design is started. If the region is not found, the interval is an interval not suitable for designing primers, and the interval is skipped.

The Sample.bed file is illustrated partially as follows:

| | | |
|---|---|---|
| 1 | 114704269 | 114704919 |
| 1 | 114704519 | 114705169 |
| 1 | 114704769 | 114705419 |
| 1 | 114705019 | 114705669 |
| 1 | 114705269 | 114705919 |
| 1 | 114705519 | 114706169 |
| 1 | 114705769 | 114706419 |

For positions found suitable for primer design, the following information is used to generate a Configure file, which is used in primer 3 to design primers.

The Configure file includes the following information:
the sequence information including an upstream flanking sequence, a sub-interval sequence and a downstream flanking sequence;
the temperature range;
the amplification length range;
the number of primers that returns;
the primer length range;
the GC content range.

The Configure file is illustrated as follows:

SEQUENCE_TEMPLATE =
TGAGTCGTATGACTAAGCCAAGAACTTCCAGTTTTTATTTTTTAAACATC
ATTTAACAAGAAAAAACATTCAACCAAATTAAAAAGAACTAGGTTGGATT
AATTTACAATAAAATAATCAACTTAAAATATCGGCCCTTCCATTTAGGGC
CAAGGAGGCCAATAGTTCCTGTTTAAACAGCAGAATTGCACAATTATTTT
TACCTATATTTGATGGCACAAAAAAATAAAAGTCTTACAACTTCCACGGA
CATCCTCGTCTGATTG

PRIMER_MIN_TM=57.0
PRIMER_OPT_TM=59.0
PRIMER_MAX_TM=62.0
PRIMER_NUM_RETURN=5
PRIMER_PICK_LEFT_PRIMER=1
PRIMER_PICK_INTERNAL_OLIGO=0
PRIMER_PICK_RIGHT_PRIMER=1
PRIMER_OPT_SIZE=20
PRIMER_MIN_SIZE=18
PRIMER_MAX_SIZE=22
PRIMER_INTERNAL_MIN_GC=35
PRIMER_INTERNAL_MAX_GC=75
PRIMER_PRODUCT_SIZE_RANGE=250-300
PRIMER_EXPLAIN_FLAG=1

After the primers are designed, the output file is formatted and used for screening downstream primers. The formatted information is as follows:

| # primer | | length | | Tm |
|---|---|---|---|---|
| ID | primer sequence | (bp) | GC (%) | (degree) |
| Id1 | ACACTTCCAAATGTC | 22 | 40.91 | 58.69 |
| | ACATCCA | | | |

The designed primers are aligned to a reference genome, and specific alignment results and non-specific alignment results are obtained. Blast is used as an alignment tool, and a seed length is designed to be 7 bp to 15 bp. Specific and non-specific results are calculated for each primer. For primers that may specifically bind to a target sequence, they have specificity. For primers with other non-specific alignment results, non-specific binding sites of each primer in each sub-interval are calculated.

For example, p1 is taken as an example for alignment.

#primer ID Aligned chromosome Identical Alignment length Mismatches Gaps start position of primer end position of primer start position of alignment binding end position of alignment binding
p1 1 100.000 22 0 0 1 22 114704270 114704291 (the specific alignment results, and other non-specific alignment results)
p1 1 100.000 16 0 0 3 18 44330811 44330826
p1 1 100.000 16 0 0 4 19 184594982 184594967
p1 4 100.000 18 0 0 1 18 187138424 187138407
p1 9 100.000 16 0 0 1 16 130274858 130274843
p1 8 100.000 16 0 0 2 17 18814823 18814808
p1 7 100.000 16 0 0 2 17 149996702 149996717
p1 7 100.000 16 0 0 2 17 154100101 154100086
p1 7 100.000 16 0 0 2 17 154384320 154384335
p1 6 100.000 16 0 0 5 20 15541460 15541445
p1 5 100.000 16 0 0 5 20 149720358 149720343
p1 15 94.737 19 1 0 2 20 71703452 71703470
p1 13 94.737 19 1 0 4 22 107793416 107793434
p1 12 100.000 16 0 0 7 22 13667731 13667716
p1 11 95.000 20 0 1 3 22 133647879 133647861

A secondary structure prediction is performed on remaining primers, including hairpin and dimer. A prediction tool may be selected from conventional online prediction tools or local open source software such as PrimerStation or MPprimer. A selected predicted temperature is 57° C. to 63° C. If the secondary structure may be formed within this temperature range, dimers in the same amplification interval are directly filtered, and dimers in different amplification intervals are labeled, and the label is represented by −N, such as C1-1 and B3-1 in FIG. 3, and N represents mutually exclusive pairs. The label is used for subsequent screening of primer combination.

Example:
two primers are able to form a dimer within the predicted temperature range, and are labeled as P1-1 and P2-1;
a dimer temperature Tm is equal to 59.20° C. (Tm=59.20° C.);

P1 AGCAAGCTAGATGCACTCCA
: :::::: : : :: ::: :: :
P2 TCGTTCGATCTACGTGAGGT

The obtained primers are divided through the optimal temperature range and the sub-optimal temperature range. The experimental temperature range is 57° C. to 62° C., and the optimal temperature in the experiment is set to 59° C. A range obtained by extending the optimal temperature by 1° C. through parameters is the optimal temperature range, i.e., 58° C. to 60° C., and thus 57° C. to 58° C. and 60° C. to 62° C. are the sub-optimal temperature ranges.

The design results are as follows:
format description: two pools including a primer pool 1 and a primer pool 2.
>1:114704170-114705019_L means XX position to XX position on the chromosome 1, L means Left (i.e., the 5′-end primer), and R means Right (i.e., the 3′-end primer).

The foregoing descriptions are merely specific implementations of the present disclosure. However, the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claim.

1. A method for designing multiplex polymerase chain reaction (PCR) primers based on iteration, comprising:
acquiring at least one target interval of one of DNA to be tested and RNA to be tested, a plurality of sub-intervals in a target interval, and at least one avoidance region in the target interval;
performing iterative design of PCR primers, avoiding the at least one avoidance region, for the plurality of sub-intervals to generate primers;
filtering and screening the generated primers according to a preset filtering condition to obtain target primers; and
combining the obtained target primers to generate a primer pool.

2. The method for designing multiplex PCR primers based on iteration according to claim 1, wherein acquiring the at least one avoidance region in the target interval, includes:
acquiring flanking information of each sub-interval in the target interval; the flanking information including at least one of guanine-cytosine (GC) contents, single nucleotide variants (SNVs) and complexities of upstream and downstream sequences of the sub-interval; and
determining a part of the plurality of sub-intervals as the at least one avoidance region in the target interval according to the flanking information.

3. The method for designing multiplex PCR primers based on iteration according to claim 2, wherein determining the part of the plurality of sub-intervals as the at least one avoidance region in the target interval according to the flanking information, includes:
if at least one of the upstream sequence and the downstream sequence has a GC content greater than 75% or less than 35%, determining a sub-interval where the at least one of the upstream sequence and the downstream sequence is located as an avoidance region in the target interval;
if at least one of the upstream sequence and the downstream sequence has an acquired SNV, a sub-interval where the at least one of the upstream sequence is and the downstream sequence is located being a mutation region, and determining the mutation region as an avoidance region in the target interval; and
if at least one of the upstream sequence and the downstream sequence has a complexity lower than a preset complexity threshold, determining a sub-interval where the at least one of the upstream sequence and the downstream sequence is located as an avoidance region in the target interval.

4. The method for designing multiplex PCR primers based on iteration according to claim 1, wherein acquiring the plurality of sub-intervals in the target interval, includes:
acquiring a length of each target interval according to a preset amplification length of the primers; and
comparing the length of the target interval with the preset amplification length;
if the length of the target interval is greater than the preset amplification length, acquiring a number of sub-intervals to be split from the target interval, and acquiring a range of a number of primers of a target fragment in the target interval.

5. The method for designing multiplex PCR primers based on iteration according to claim 4, wherein after comparing the length of the target interval with the preset amplification length, the method further comprises:
if the length of the target interval is less than or equal to the preset amplification length, determining that the target interval is amplified by using a pair of primers.

6. The method for designing multiplex PCR primers based on iteration according to claim 2, wherein performing the iterative design of PCR primers, avoiding the at least one avoidance region, for the plurality of sub-intervals to generate the primers, includes:
according to a position of an avoidance region and flanking information of the target interval, determining whether a sub-interval is adjacent to the avoidance region;
if the sub-interval is adjacent to the avoidance region, shifting the sub-interval to skip the avoidance region; and
if the sub-interval is not adjacent to the avoidance region, maintaining a position of the sub-interval.

7. The method for designing multiplex PCR primers based on iteration according to claim 6, wherein filtering and screening the generated primers according to the preset filtering condition to obtain the target primers, includes:
aligning the generated primers to a genome to sort binding sites of the primers and the genome in reverse order and screen out a primer pair with least non-specific binding in each sub-interval, so as to obtain remaining primers after screening; and
filtering dimers in the remaining primers in the same amplification interval directly, and labeling dimers in the remaining primers in different amplification intervals.

8. The method for designing multiplex PCR primers based on iteration according to claim 7, wherein combining the obtained target primers to generate the primer pool, includes:
selecting primers within a preset temperature range according to the obtained target primers;
splitting a combination of primers with dimer mutual exclusion into different primer pools; and
selecting primers without mutual exclusion for combination to generate the final primer pool.

9-13. (canceled)

14. A computer device, comprising a memory and a processor, wherein the memory has stored therein a computer program capable of being run on the processor; when executing the computer program, the processor performs:
acquiring at least one target interval of one of DNA to be tested and RNA to be tested, a plurality of sub-intervals in a target interval, and at least one avoidance region in the target interval;
receiving the at least one avoidance region in the target interval, and performing iterative design of PCR primers, avoiding the at least one avoidance region, for the plurality of sub-intervals to generate primers;
receiving the generated primers, and filtering and screening the primers according to a preset filtering condition to obtain target primers; and
receiving the obtained target primers, and combining the target primers to generate a primer pool.

15. A non-transitory computer-readable storage medium having stored therein a computer program that, when executed by a processor, causes the processor to perform the method according to claim 1.

16. The method for designing multiplex PCR primers based on iteration according to claim 8, wherein the preset temperature range includes an optimal temperature range and a sub-optimal temperature range.

17. The computer device according to claim 14, wherein when executing the computer program, the processor further performs:
acquiring flanking information of each sub-interval in the target interval, the flanking information including at least one of guanine-cytosine (GC) contents, single nucleotide variants (SNVs) and complexities of upstream and downstream sequences of the sub-interval; and
receiving the flanking information, and determining a part of the plurality of sub-intervals as the at least one avoidance region in the target interval according to the flanking information.

18. The computer device according to claim 17, wherein when executing the computer program, the processor further performs:
if at least one of the upstream sequence and the downstream sequence has a GC content greater than 75% or less than 35%, determining a sub-interval where the at least one of the upstream sequence and the downstream sequence is located as an avoidance region in the target interval;
if at least one of the upstream sequence and the downstream sequence has an acquired SNV, determining a sub-interval where the at least one of the upstream sequence and the downstream sequence is located being a mutation region, and determining the mutation region as an avoidance region in the target interval; and
if at least one of the upstream sequence and the downstream sequence has a complexity lower than a preset complexity threshold, determining a sub-interval where the at least one of the upstream sequence and the downstream sequence is located as an avoidance region in the target interval.

19. The computer device according to claim 14, wherein when executing the computer program, the processor further performs:
acquiring a length of each target interval according to a preset amplification length of the primers;
comparing the length of the target interval with the preset amplification length; and
if the length of the target interval is greater than the preset amplification length, acquiring a number of sub-intervals to be split from the target interval, and acquiring a range of a number of primers of a target fragment in the target interval.

20. The computer device according to claim 19, wherein when executing the computer program, the processor further performs:
after comparing the length of the target interval with the preset amplification length, if the length of the target interval is less than or equal to the preset amplification length, determining that the target interval is amplified by using a pair of primers.

21. The computer device according to claim 17, wherein when executing the computer program, the processor further performs:
performing the iterative design of PCR primers for the plurality of sub-intervals;
determining whether a sub-interval is adjacent to an avoidance region according to a position of the avoidance region and flanking information of the target interval;
shifting the sub-interval to skip the avoidance region if the sub-interval is adjacent to the avoidance region; and
maintaining a position of the sub-interval if the sub-interval is not adjacent to the avoidance region.

22. The computer device according to claim 21, wherein when executing the computer program, the processor further performs:
aligning the generated primers to a genome to sort binding sites of the primers and the genome in reverse order and screen out a primer pair with least non-specific binding in each sub-interval, so as to obtain remaining primers after screening; and
filtering dimers in the remaining primers in the same amplification interval directly, and labeling dimers in the remaining primers in different amplification intervals.

23. The computer device according to claim 22, wherein when executing the computer program, the processor further performs:
screening a temperature range adapted to the obtained target primers; and
distinguishing a combination of primers with dimer mutual exclusion and splitting the combination of primers with dimer mutual exclusion into different primer pools.


SEQUENCE LISTING (1 sequence)

SEQ ID NO: 1 - DNA, 6247 bp, Artificial Sequence
  designed by a method for designing multiplex PCR primers based on iteration to be used for exons of four genes, KRAS, NRAS, BRAF and PIK3CA which are obtained through a GRCh38 genome annotation file in a format of General Feature Format version 3
tccaacacttccaaatgtcacaaggatgtccgtggaagttgttgtgtggcacattagactgcaccatcctttaaactgctgtgaacatacaagggctattggcacactgaggtcctccatcttctgcctccaaatcctcataggtatgaagaggtcagttagcacaaacgtgagaaggggattgaaggccccaggagaaagatgaaactgaagtcacagacgtatctcagacatgagctctaaagtagcaactgccgtttgcggtttggttctctgacaccctggtcctgacttcacaccagcactcctccaaatttgtgtccgttgagctagcttgtggtaacctcatttccccaccacacccccaggattcttattctggcatcagtgaaacgcatgactcgtggttcggagggtcagcgcaaccaaatgatgtatgttgagggcccatctcttgcagaattcatgctatccagtactgttgtcaccattgcacaagcctagaatgcctacttgggaggtgcctgaactagttcacagcacctgtttatttgtacccaggatgccagtcaccacacaagtccactggatagggttctgtcctaccttccacatgccccatcagattgtgctgagcttgacatggctagttctcttaacactggatccaaactgccctagtcccagcaaccattttggggctatctgggtctgccttaacaggaccaattgtgaatgttggtgtgatggttgccaccttgttacctttcttgcacatggctttccctgcatggcattagcaaagacttccagactgtgtttctcccttaattcattcactcgccgccgagctcgattttcctaggcggacggagaaaggagtcgggctctactcacgctcggggagcctcatctctgtcttgatgacctcacggaggcattctaaagacagagttccctgtttgcaatgctttggcaattctggtgatgattgatcttgtgcttcaacgacattcacgtaggttgcacacccttggcatcatcagtcttagcaaatggaaaggcaaagtctttggccagtacctcatggagcttctcgggatacagaccatctttgccattataactgtgccacctgggctacttcatctctttcgaggttttgctgttcgggccaaaagaaaaagtgcccaacaggaaggggtacacagtgagaggagacagaaagccctgtgtaaaggctttgagtggacaaggttgccttactggttaccttggtcagggaacatctggaatgtccatcgtctttcaccatgtgtgacatttgagcaaagacctgttgtccagccaccatgatgtgcacaatccatgaacagcaacaagcagtcaaaaacccacttgcagttttggaagcagtcatttggggtgcaaaacaatgcatgaggctcaggcactatccagaatgtgtcctgtgctcaatccaggtgccacatcaaaaaccctgatattgaaaaggctgggcccagccttttcaatatcagggcaatgtttggttctttgcctcattttgatgaaaacagctgctgattgagtatgtgggtccagccggctggacccacatactcaaaaagaaagcaggcttcccagacctcagaaggaaaatgggaaaagatgcctcttgactagggcaagtgacaggtgtgagccatttttctgcctccttgccaacaggttggagaacttggcctttgcagtataccacaggacttcaccagaggaatgggtgaagcactctgtcaacacacacacacaagggtagacagtcactaaccatgagatctagtcccaccctctcttcagcagtcctgtccctcactgatttctaaggaatggggcacttaacgtgggaggagtccaaaaggctgacaccaagctggaagacttctccatgcagtcacgtgcccagtgaaaagatggagcatctccctttcctctccgtcctccgtgtttcctttgggctagccacttacgttggtcactgcgttgcatttttgtgtcgggagaagcagatggtttgaggacagaaacccaactccttgtctttgcccaaacaaaagtgggaggtctcaaggcatcctttggcagcagtactctggaaattgtgaagagagcagccactagagcctcacctacaccattaccggtttaggggtacgttttcgtcatgttctagagctcctgcagcactgtcaacctgaaaggggtgagatattcgggaactgtggctgcgtcactgatacgcagccacatactgtctaagagagacctgggcttgaacacactgccctgctgatgtaaagtcatagcaaagaaagcggatgttagtgtggatccagcaagaaacccccacaaaactgctttggtcactaggggaaagagctctctgctgccagtgtttcttggcctcaagtaatccacccggctgcagttgactttctccggcatgatgaaggctcacagtggcctttctctcaagtgagtagagggctcaggttttaggagggtgtggctaagctctgtaaagagacccctgaccttgtgcggcaaatctttaacccctcacactgaccaccacattacagtcgtggcacgtttgacatgagtttggtcacctgcactcaaaaaccaaagaacacttgtgtggttaccacacaagtgttctttggttcgcctctattgagctgctgggatggccagagttaacccttgatgtggcaagggcttactaaagcaggctgtggtatccttttcgaggccagagtcctttgctaatagttgctaccactgggtggtatggagttagggctatgagcaaaaagcagtgccgtagactgcctcattacctggctcatcatcagagagaaaccagaagcgccccaaattctcaccagtctggaagaaaaaccctcacagactcttgaacccccggttttcaggcctacagtatttcttcaggcaggtgtggagttacagtccgacttcccccttagttctgatgttgacacaggaacatatagaggccgccgcctctttccaaaataagctctccgcctcccttctccatttagggccaaggaggatggtgcgttctgcttcttggctcacaagctctggaatggaaattaagtgcctggccagcgcacaactctgcaggttcattgaggatgtctcagctgagaacgtgatactgagttttccagtaccagaaagctctacatttgcatcagtaagctaggggaaacaaaactttcacagtgaagtgcctagagcagatgccagtttagagatctcacagagaagctcctgcgaagtcaggaccagggtgtcaggcataccctattcctggctggatcacatctctaccagagtgccactgtacccagcctaatggttttagaaacttcagcagctcaaactggtggtggttggaggctgcataaaagattcactggttgtgggacatatgcagtgtgtagctctgtgggggtgtgccagcagttaccttaaagctgaacatgccccacaaagtttcttgcagtgcttatgaggggataccctacctaaacagtgttccaactgaaactcttcgatcaagctaatcgcttgaacctgggagaggtggctgatgctttgaacatggagatatccacagcagcaagatggcatggtgaggtgaattcacctcaccatgccatctcacggtcatccagtgttgtcagagcagtctgacacagggcatggtcactctccccaaaagctaacagtctgcatggagccctgtacacatgaagccatcgagcaatgccctctcaagagatgacaaaagttgtggacaggtggaccctgacatactcccaattgtggtagttggagctggtacaacaccctcactactgcaccgctcacatcgtgtaaacaaggaatagtgatggtggccaccggccacacaaactatgatttgatgcccccaagaatcctacggttgcctactggttcaatttgggaatgatctggcagcgcgagagtgagattccgtcttgagtctatcgagtgtgtgcaatggggtcttgctttgttgctccttttggggaagaaaagtgtagagagaaggtttgactgccatggttctttcctgtctctgaaatcctgctcagtgatttcagagaggcagtgttttagatggctcacagggtcagtttctgcatggactacctgaaactcatggtggttctcctgtttgagaatgtcagttggccagaatttctgttaccaactcttccttaccatccccatggggaaaggcagtaaaggtactgatgtagtgtgtggctgttgggaattggagatcgtcacatttgtgcattcttgggctccacattcgaaagaccctagcctggcataacatgaaattgcgcaaggctgttattgcattaggtgtgggagcccagaatttcccttctccttctgcaattgaactgaggggtttaaggaatcaggaagactcattcccaagtagtcagagcagagtcccttgttcactactcatgagtagtgaacaagggactctgcatttttggcactcgggtaggctctacagttttattgggccttcaccaggttcccattttctccatgcttgccaactctgaacccctcacattcccctcttctgacagaataatacagctgggcaacaaggcatgccaactctgtgtgtacacactgcttgcttactgtatacgcacaagggacagtcccttgtgcgtatacagtaggtctgaggactgaatcacaattcgaaccacccttttggcgcgtgatgttacagactgcactacaagtgtgtgccaccacttcctccacagcacttagtaaaggcatctcccactggtgagaaaccggagaagtgtcccccgcccactccccaatatcaaagtgcctggcccatagtaagcccactaagcagtaaccaacacctgctcaggtcccttcatttgcaaagcaggtatagagaggtaccaagaaaaaggaaatccccccaggcgatatcatgaaggcctgtgagggtgggaaataaactgactccaccaaaacacacgtgggacacagccaatgtaagcctcactcattgagttgccgacctgtggtccatgttgctagcacaagatacaagaaccacagcatgctgtagtttagggttgaagtctttgaagagcctatgggagtagccagagatggaaggtttacactgtcatgccaagcctactttcgaaaccacattgcagggcaccgcaccaaaccagaagaggacctagacttttgccactcacactacagaaggcactgccaggagccaggcttagcataaggctgcacatgttctaccttgcagccactaaaagctcacttcaagtccttggctatagcttgcagctgaattcccagacaggtgccagcactatctagccttggctgatcatctgatgggggcagacttgtatgctcgtggagccttcaagtagccaccatccctgtgattttagacacctcaggctgacgtgggaggatctctggcatagacacatcgagtccctcggatggcatataccagaacaatgagtttctgagtggccccctcatttgggtagcttaagtgagtgattttcctggatgacatgtctcatgttctgtcctcctgcgtttgcaacaaaatttgacttgggttagataaggcaaactgacccggaacagaaagtaaagcccaaaaacacccatccaggcactgctcaaccctcatgaagcgtgggacgaggatacctgtcacgtgttgctattactgccaaggcaccagaagtcatcagaagcatccttatgttcctggacatcatgggctattccacaaagcgagtcccgactgctgtgaagacgggactcgagtgatgatacagaaagcttgtggcagtggatgcgctgtcttcggaaataagcatgtcactgaagagcaccacccaataccacaggaagacaaatgttggcctctaggaggggacactgatatttcctggctgcatgacctctgagtatgaagagcaagctagatgcactccatcatccccacgaccatgtaacagcgccttcctacgtaaaccatgtccccgttgaacagagcttcccccaatcccctcag